(19) United States
(12) Patent Application Publication
(10) Pub. No.: US 2003/0158555 A1
(43) Pub. Date: Aug. 21, 2003

(54) Surgical screw and tool for its insertion

(76) Inventors: Roy Sanders, Tampa, FL (US); Karen Gallen, Huntingdon Valley, PA (US); Jukka Rouhiainen, Helsinki (FI); Kimmo Lahteenkorva, Tampere (FI)

Correspondence Address: KENYON & KENYON, 1500 K STREET, N.W., SUITE 700, WASHINGTON, DC, 20005, US

(21) Appl. No.: 10/075,597

(22) Filed: Feb. 15, 2002

(51) Int. Cl. A61B 17/56

(52) U.S. Cl. 606/73

(57) ABSTRACT

A surgical screw system using a surgical screw that is at least partially bioabsorbable in vivo, the screw having a recess (or optionally recesses) in its head for receiving an inserter instrument, wherein the recess has a rotational symmetry around the longitudinal axis of the screw. The recess comprises an odd number of rounded lobes extending away from the center of the head. The inserter for the screw comprises a distal end that matches the shape of the recess in the screw and can be slidably engaged with the recess.

### FIELD OF THE INVENTION

The present invention relates to an at least partially bioabsorbable surgical screw and instrument for its insertion. The surgical screw of the present invention is intended to be used for bone-to-bone fixation, soft tissue-to-bone fixation, or the fixation of implants or prostheses to bone and/or to soft tissue.

### DESCRIPTION OF THE PRIOR ART

A variety of surgical screws and/or insertion instruments are known, e.g., from the following publications:

U.S. Pat. No. 6,077,267 discloses a bone screw comprising a threaded shank and a head, which is integral with the shank. A plurality of separate drive receivers are disposed on the circumference of the head.

U.S. Pat. No. 5,470,334 discloses a bone screw fabricated from bioabsorbable material. A drive recess is formed in the body to extend longitudinally. The drive recess defines a plurality of radial force receiving surfaces for receiving concentric forces from the driver applied perpendicularly to the force-receiving surface.

U.S. Pat. No. 5,169,400 discloses a bone screw comprising an insertion channel which is open at the top and which is arranged concentrically with the shaft and extends most of the length of the shaft. The cross-section of the channel is non-circular and corresponds to the cross-section of the screwing-in tool.

U.S. Pat. No. 5,968,047 discloses a bone screw comprising a recess for a driver. The recess can be for example hexagonal or quadratic.

U.S. Pat. No. 5,961,524 discloses a tapered bone screw, which is self-centering and self-aligning. The screws are inserted in a pilot hole and then turned to lock the screw in its final position.

U.S. Pat. No. 6,096,060 discloses a soft tissue anchor comprising a hole which extends through the anchor. The hole is preferably a triangle with rounded corners.

U.S. Pat. No. 6,283,973 discloses a screw for use with soft tissue grafts comprising a passageway extending all the way through the screw body. The passageway has a polygonal shape, preferably a square.

U.S. Pat. No. 5,019,080 discloses a prosthetic fastener comprising a recessed socket having a hexalobular shape.

U.S. Pat. No. 6,269,716 discloses a fastener having a threaded shaft and a star-shaped head. A mating driver snugly fits around the star-shaped head of the fastener, to thereby apply torque to the perimeter of the star-shaped head.

WO 90/08510 discloses a screw comprising an axial bore of circular cross-section and a counterbore of polygonal cross-section.

FI 891974, the disclosure of which is hereby incorporated by reference, discloses a bone screw comprising a longitudinal channel, which extends at least partially to the shank of the screw. The cross-section of the channel can be for example a triangle, square, rectangle, pentagon, hexagon, cross-shaped or star-shaped.

EP1101459 discloses a bioabsorbable screw having a tapered profile. The screw includes a head provided with a specially designed drive socket with radially extending slots at its outer end for receiving corresponding protrusions on the shaft of screwdriver. The socket has a taper corresponding to the tapered outer profile of the screw.

Inserting surgical screws, such as bone screws, requires extreme accuracy and directional control and therefore it is important that the screw/insertion instrument combination be firm and stable. Further, the screw must be strong enough to withstand both the forces to which it will be exposed during the healing of the wound it is repairing and the torsional forces to which it is subjected during insertion. It is particularly difficult to develop a bioabsorbable screw that can fully withstand both types of forces.

The torsion resistance of non-reinforced biostable and absorbable screws is so small that the screws break or fracture easily upon stress overload. As an example, the screw-head can torsionally shear off when screws are turned into the bone (see e.g. the publications Unfallchirurg 88, 1985, 126-133, Gay and Bucher, and Dtsch. Z. Mund-Kiefer-Gesichts-Chir. 9, 1985, 196-201, Ewersu and Förster).

The module of fiber-reinforced biostable, partially absorbable or totally absorbable screws made of polymer composites is clearly less than the module of metallic screw materials. The modules of polymer composites are typically between 5 and 50 GPa, whereas those of implant steel are around 200 GPa. As a result, the polymer composites are considerably more flexible than steel. This is an advantage in relation to the fracture healing (see, for example, the Thesis of P. Axelson, “Fixation of cancellous bone and physeal canine and feline fractures with biodegradable self-reinforced polyglycolide devices”, Veterinary University, Helsinki), but the flexibility of the materials from which these screws are made makes them more difficult to insert. For instance, such screws cannot easily be inserted simply by placing a screwdriver in a slot located in the middle of the screw head because the screwdriver tip easily starts to rotate in the screw-head slot because of the small boundary surface between the tip and the slot. However, this kind of head is very handy in steel screws (see e.g. AO/ASIF Instrumentation, Sequin and Texhammar, Springer Verlag, Berlin, 1981). Likewise, polymer screws may be prone to being damaged during insertion if the torsional stress applied by the insertion instrument is concentrated in a relatively small area of the screw. Steel screws are less likely to suffer such damage.

To allow for ease of insertion, polymer screws have possessed heads with large cross-sections or have been short (see e.g. the Thesis of J. Leenslag “Poly(L-lactide) and its Biomedical Applications”, University of Groningen, 1987). Increased head size is not preferable, since it is generally beneficial to reduce the size of implant devices, to reduce trauma to the patient and also to reduce the likelihood of any negative side-effects resulting from the bioabsorption of the device in vivo. Limitations on the length of the screws are not preferable, since it reduces the situations in which the screws may be effectively used.

Alternatively, polymer composite screws have been turned with a screwdriver that is applied to the outer surface of the screw, or must be externally supported on the outer surface of the screw. One disadvantage of this type of screwdriver/screw combination, however, is that it increases instability, making the screw difficult to advance. This complicates insertion of the screw, the screw head might disengage from the driver causing delays to the operation, and in the worst case the head of the screw may be shattered.

Therefore, an object of the present invention is to provide a surgical screw made of at least partially bioabsorbable polymer or polymer composite, in which the above-mentioned shortcomings of known bioabsorbable screws can be eliminated effectively.

It is a further goal of the present invention to provide a bioabsorbable surgical screw, which can, because of better torsion resistance and minimal concentration of torsional stress, be screwed into tissue, like bone, more tightly than the corresponding screws and that way achieve a tighter fixation than with the known screws.

It is a further goal of the present invention to provide a screw/insertion instrument combination that is stable during the insertion.

It is a further goal of the present invention to provide an insertion instrument for the surgical screw that holds the screw without any external aid.

It is a further goal of the present invention to provide a surgical screw that will biodegrade and is of minimum mass, thereby reducing the risk of post-operative complications due to the presence of the fastener in the patient.

It is a further goal of present invention to provide a surgical screw that is easy and quick to use, thereby reducing the length and difficulty of surgical procedures.

It is a further goal of present invention to provide a bioabsorbable screw that is naturally degradable and absorbable by the body during the healing period, thereby obviating the need for a secondary surgical removal procedure.

It is a further goal of the present invention to provide a screw that can be suitable for use with a guide wire.

### SUMMARY OF THE INVENTION

The surgical screw of the present invention is primarily intended for the fixation of bone fractures, osteotomies, arthrodesis, lesions of tissues such as cartilage and ligament, and for affixing implants or prostheses. The surgical screw is made of at least partially bioabsorbable material, however in certain preferred embodiments, the material of the screw may contain also other materials or substances, for example pharmaceuticals.

The screw comprises a longitudinally extending shank and a head. The shank is at least partially threaded but in a preferred embodiment it is entirely threaded. In a preferred embodiment, the head has a tapered distal surface leading to the shank and a proximal surface, which forms a substantially perpendicular plane with respect to the longitudinal direction of the shank. In a preferred embodiment, the head's circumference is circular in the plane perpendicular to the longitudinal direction of the shank and its diameter may be greater than the diameter of the shank. In this embodiment, the distal surface of the head stops the screw at the final stage of insertion by contacting the proximal surface of the material into which the screw is being inserted. In another embodiment, the diameter of the head can be the same as the shank, and the threads may cover at least partly the circumference of the head.

In a preferred embodiment, the proximal surface of the head includes a recess for receiving a screw driver or other instrument for inserting the screw, which is comprised of a plurality of lobes, which are arranged in rotational symmetry around the center of the head. Rotational symmetry means that the screw can be rotated a certain number of degrees, for instance 120 degrees, and the recesses will look the same as they did in their original position. The head includes preferably an odd number of the lobes. The lobes may be separate (in which case there are multiple recesses in the head) or incorporated to form a continuous pattern, such as a cloverleaf. The lobes may be located partially one upon the other. Generally the lobes are located so that they preferably divide the plane of the screw head to equal parts, thereby the area between lobes is as large as possible. The size of the lobes of the drive recess can vary or all the lobes can be equal in the size.

The lobes are preferably circles, ovals or rectangles with rounded edges but also other shapes are possible. The lobes have outer ends which have no sharp angles. The shape of the recess is chosen so that it prevents the screwdriver tip from rotating inside the screw in the drive recess in relation to the screw wall when turning the screw. At the same time the stress concentration is minimized due to the odd number of the lobes and the rounded shape of the outer ends of the lobes. The use of curved lobes thus allows for greater torque to be applied to the screw without causing damage to the screw head.

Because the main contact by the inserter instrument is made with the walls surrounding the recess, it is advantageous for the circumference of the recess to be long. This increases the contact surface between the screw and the inserter, thereby dispersing or distributing the turning force over a wider area. In addition, it is advantageous if the cross-section of the recess is small. Thus, the mechanical properties of the screw are less affected in that the screw is less likely to be weakened by the presence of the recess.

The recess may extend through the head along the longitudinal axis of the screw and into the shank. In certain embodiments, however, the recess is not so deep as to enter the shank of the screw. Generally, the depth of the recess is at least 3% of the total length of the screw. In a preferred embodiment of the present invention, the recess extends the entire length of the screw. In these embodiments, the turning force disperses along the entire length of the screw, which allows the screw to be turned more easily and more forcefully without being damaged, thereby allowing for secure and tight insertions of the screw in vivo.

In other embodiments of the present invention, a bore extending deeper into the screw than the lobes may be present in the bottom of the recess. The bore is coaxial with the longitudinal axis of the shank and it may have circular cross-section. In certain embodiments, the depth of the bore is 5-10% more than the depth of the recess. In certain other preferred embodiments, in addition to the recess, the screw has a central bore running entirely through the head and the shank, which may aid in the insertion of the screw.

The lobes forming the recess are preferably made by machining, preferably by a milling tool. The use of the milling technique is advantageous because the lobes can easily be formed in different sizes and shapes, and can be formed in the same process step as the threads on the shank. In embodiments having a central bore in addition to the recess, with the milling technique, the central bore is first formed and then the milling tool is arranged to mill the lobe according to a predetermined pattern.

The screws of the present invention can be made of biocompatible and bioabsorbable polymers, copolymers, or polymer mixtures. In certain embodiments of the present invention, the screws are also reinforced with bioabsorbable fibers.

Table 1 lists a variety of known absorbable (biodegradable) polymers which can be used, alone or in mixtures, as raw materials for devices of the present invention both as matrix (or binder polymers) and/or reinforcement elements.

Table 1. Biodegradable Polymers

1. Polyglycolide (PGA)

Glycolide copolymers

2. Glycolide/lactide copolymers (PGA/PLA)

3. Glycolide/trimethylene carbonate copolymers (PGA/TMC)

Stereoisomers and copolymers of PLA

4. Poly-L-lactide (PLLA)

5. Poly-D-lactide (PDLA)

6. Poly-DL-lactide (PDLLA)

7. L-lactide/DL-lactide copolymers

L-lactide/D-lactide copolymers

Copolymers of PLA

8. Lactide/tetramethylene glycolide copolymers

9. Lactide/trimethylene carbonate copolymers

10. Lactide/δ-valerolactone copolymers

11. Lactide/ε-caprolactone copolymers

12. Polydepsipeptides (glycine-DL-lactide copolymer)

13. PLA/ethylene oxide copolymers

14. Asymmetrically 3,6-substituted poly-1,4-dioxane-2,4-diones

15. Poly-β-hydroxybutyrate (PHBA)

16. PHBA/β-hydroxyvalerate copolymers (PHBA/PHVA)

17. Poly-β-hydoxypropionate (PHPA)

18. Poly-β-dioxanone (PDS)

19. Poly-δ-valerolactone

20. Poly-δ-caprolactone

21. Methylmethacrylate-N-vinylpyrrolidone copolymers

22. Polyesteramides

23. Polyesters of oxalic acid

24. Polydihydropyranes

25. Polyalkyl-2-cyanoacrylates

26. Polyurethanes (PU)

27. Polyvinyl alcohol (PVA)

28. Polypeptides

29. Poly-β-maleic acid (PMLA)

30. Poly-β-alkanoic acids

31. Polyethylene oxide (PEO)

32. Chitin polymers

Biodegradable polymers other than those set forth in Table 1 can also be used as raw materials for implants, devices and parts thereof of the present invention. The screws of the present invention can be manufactured using either one polymer or a mixture of polymers.

Screws of the present invention can be reinforced with polymer fibers or fiber mixtures (such as mixtures of bioabsorbable fibers) which have been made of the above bioabsorbable polymers, copolymers or mixtures thereof. Also other biocompatible fibers, such as carbon fibers, aramide fibers, glass fibers, aluminum oxide fibers, and biostable ceramic fibers may be used as reinforcement for the screws of the present invention. Degradable glass fibers, such as tricalcium phosphate fibers, can also be used as reinforcement.

Screws of the present invention can also be reinforced through self-reinforcing techniques. A self-reinforced absorbable polymeric material is uniform in its chemical element structure and therefore has good adhesion between the matrix and reinforcement elements. The material has excellent initial mechanical strength properties, such as high tensile, bending or shear strength and toughness, and therefore can be applied favourably in surgical absorbable osteosynthesis devices or as components or parts of such devices, such as screws.

Self-reinforcement means that the polymeric matrix is reinforced with reinforcement elements or materials (such as fibers) which have the same chemical element percentage composition as does the matrix. By applying self-reinforcement principles, the high tensile strength of the fibers can be effectively utilized, when manufacturing macroscopic samples.

When strong oriented fiber structures are bound together with the polymer matrix which has the same chemical element composition as the fibers, a composite structure is obtained which has excellent adhesion between the matrix and reinforcement material and therefore also has excellent mechanical properties.

The material that will form the matrix is subjected to heat and/or pressure in such a way that it allows the development of adhesion between the reinforcement fibers and the matrix. There are alternative methods which can be applied in manufacturing self-reinforced absorbable osteosynthesis materials of the present invention. One method is to mix finely milled polymer powder with fibers, threads or corresponding reinforcement units which are manufactured of the same polymer material or of its isomer with the same chemical element percentage composition, and to heat the mixture under such conditions and using such temperatures that the finely milled particles are softened or melted but the reinforcement unit structures are not significantly softened or melted. When such composition is pressed to the suitable form, the softened or melted particles form a matrix phase that binds the reinforcement units together and when this structure is cooled, a self-reinforced composite with excellent adhesion and mechanical properties is obtained.

The self-reinforced structure of certain embodiments of the present invention can also be obtained by combining together the melt of an absorbable polymer and fibers, threads or corresponding reinforcement elements of the same material, forming the mixture of the polymer melt and reinforcement elements into the desired form and cooling the formed polymer composite rapidly so that the reinforcement elements do not significantly lose their oriented internal structure.

One can also manufacture the self-reinforced absorbable material of the present invention by heating absorbable fibers, threads or corresponding structures in a pressurized mold under such circumstances that at least part of these structures are partially softened or melted on their surface. Under the pressure the softened or melted surface of fibers, threads or corresponding structures are coalesced together and when the mold is cooled, a self-reinforced composite structure is obtained. By a careful control of the heating conditions it is possible to process composite samples where the softened or melted surface regions of fibers, threads or corresponding units are very thin and, therefore, the portion of oriented fiber structure is very high, leading to materials with high tensile, shear, bending and impact strength values.

Screws in accordance with the present invention can be manufactured of polymers, copolymers, polymer mixtures and possible degradable and/or biostable reinforcement fibers by various other methods, which are used in plastics technology as well, such as injection molding, extrusion with fibrillation and forming or compression molding wherein the particles are formed from raw materials with aid of heat and/or pressure.

Screws in accordance with the present invention also can be manufactured from the above raw materials by so-called solution techniques wherein at least part of the polymer is dissolved or softened by a solvent and the materials or material mixture are affixed to an article through the application of pressure and possibly gentle heat whereupon the dissolved or softened polymer glues the material to the article. The solvent is then removed by evaporating.

Screws of the present invention may also contain various additives and adjuvants for facilitating the processability of the material such as stabilizers, antioxidants, or plasticizers; for modifying the properties of thereof such as plasticizers, powdered ceramic materials, or biostable fibers such as aramide or carbon fibers; or for facilitating the manipulation thereof such as colorants.

In a preferred embodiment, screws of the present invention contain some bioactive agent or agents, such as antibiotics, chemotherapeutic agents, wound-healing agents, growth hormones, contraceptive agents, and anticoagulants such as heparin. Such bioactive devices are preferred in clinical applications, since, in addition to mechanical effect, they have beneficial biochemical effects in various tissues.

### BRIEF DESCRIPTION OF THE DRAWINGS

FIGS. 1a and 1b show side views, with partial cutaway views, of embodiments of surgical screws of the present invention;

FIG. 1c shows a cross-sectional view of one embodiment of a surgical screw of the present invention;

FIG. 1d shows a top view of the head of a screw of the present invention;

FIGS. 2a-2d show top views of the head of various embodiments of screws of the present invention;

FIG. 3 shows a side view of an installation instrument of the present invention;

FIG. 4 shows a bottom view of the distal end of the installation instrument of FIG. 3; and

FIG. 5 shows an installation instrument of the present invention being inserted into a screw of the present invention.

### DETAILED DESCRIPTION OF THE PREFERRED EMBODIMENTS

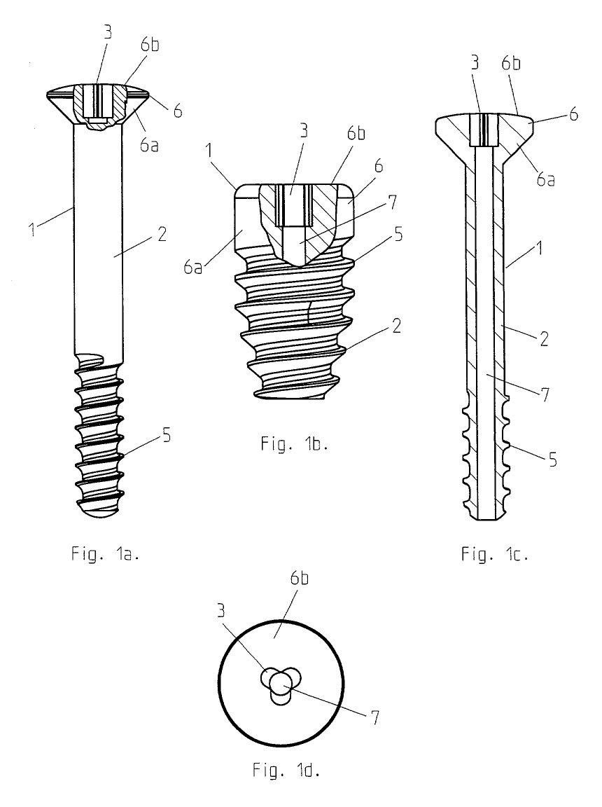

As seen in FIG. 1a, a surgical screw 1 comprises a shank 2 and a head 6. The shank 2 is at least partially threaded with threads 5. In other embodiments (such as that shown in FIG. 1b) the threads 5 can cover the whole shank 2. The head 6 has a tapering distal surface 6a and a proximal surface 6b. The distal surface 6a of the head 6 leads to and abuts the shank 2. The proximal surface 6b of the head 6 is substantially perpendicular to the longitudinal axis of the shank 2. The head 6 comprises a recess 3 which is coaxial with the longitudinal axis of the shank 2. As shown in FIGS. 1b and 1c, a central bore 7 may extend further through the screw 1. Recess 3 is designed to receive the distal end of an insertion instrument, such as a screwdriver. Preferably, the distal end of the insertion instrument is inserted into the recess 3 by simply sliding it into the recess. When the distal end of the instrument is so inserted, the instrument holds the screw 1.

To turn the screw into the tissue to be repaired (for example into a threaded drill channel), the surgeon inserts the distal end of an insertion instrument into the recess 3, aligns the screw 1 with the channel into which the screw is to be inserted, and rotates the screw about its longitudinal axis. When the screw is fully inserted, the distal surface 6a of the head 6 will contact or embed in the proximal surface of the material into which the screw is being inserted and will stop the forward progress of the screw.

The torque force opposing the turning of the screw increases rapidly as a result of the greater cross-section of the head 6 in comparison with that of the shank 2 when the head comes into contact with the tissue as the screw turns deeper into the tissue. The depth of the drive recess 3 may be varied according to the screw size and force needed to insert the screw.

Another embodiment of a screw of the present invention can be seen in FIG. 1b. The diameter of the head 6 is roughly equal to the widest diameter of the shank 2 comprising threads 5. In other embodiments, the threads 5 may cover the circumference of the head 6 at least partially.

FIG. 1d shows a top view of the upper part 6b of the head 6 of a screw of the present invention. The head comprises a recess 3 having three lobes which are arranged in rotational symmetry around the center of the longitudinal axis of the shank. In FIG. 1d, the rotational symmetry is 120 degrees, since the recess will look the same if rotated around the longitudinal axis of the screw 120 degrees. The lobes are arranged to form a cloverleaf design. The length of the recess may vary, but preferably is at least 3% of the total length of the screw. The screw also has a central bore 7 in the middle of the cloverleaf that extends deeper than the recess 3. The shape of the cloverleaf is advantageous because it allows a firm engagement with the insertion instrument and, when torque is applied to the instrument, stress concentration is minimized.

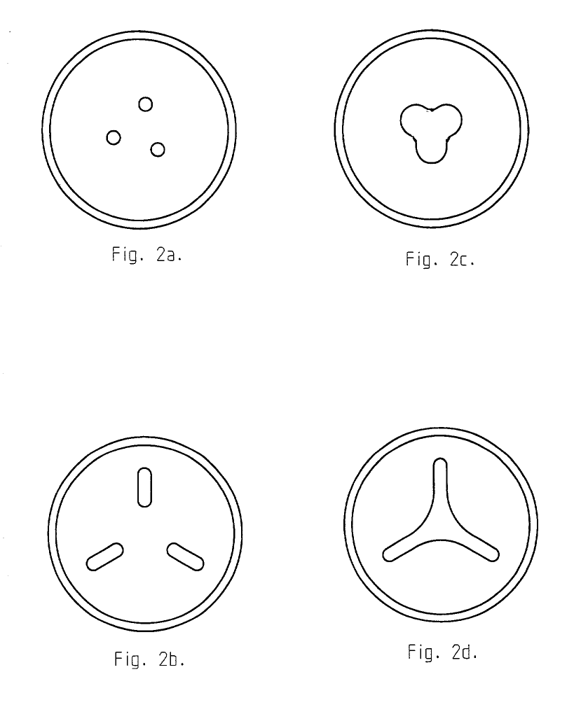

FIGS. 2a-2d show other possible geometries for the recesses in the screws of the present invention for receiving the insertion instruments. FIG. 2a shows three separate lobes having a circular shape located in rotational symmetry around the center of the longitudinal axis of the shank. FIG. 2b also shows three separate lobes located in rotational symmetry around the center of the longitudinal axis of the shank, but the shape of the lobes is a rectangle with rounded edges. FIG. 2c shows three lobes arranged in a cloverleaf pattern similar to that of FIG. 1d, but without a central bore. FIG. 2d shows a three-armed recess in which the lobes are elongated and have rounded ends. In each of FIGS. 2a-2d, the rotational symmetry is 120 degrees. The rotational symmetry need not be 120 degrees, however. If a different number of lobes or recesses were present, then a different rotational symmetry (i.e, 72 degrees for five equally spaced lobes) would exist.

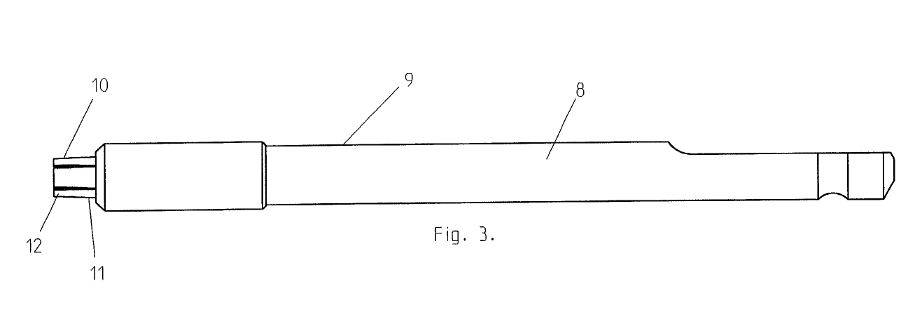

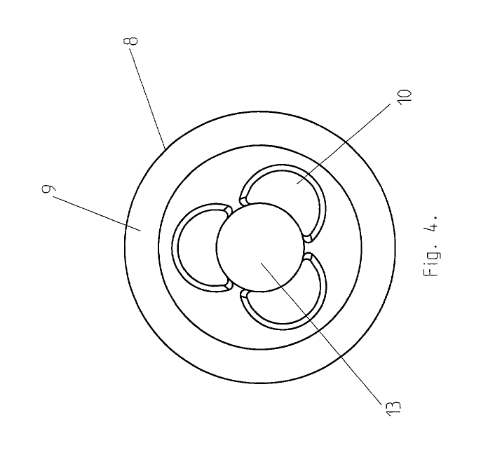

FIGS. 3 and 4 show a surgical insertion instrument 8 of the present invention, preferably made of stainless steel, comprising an elongate, cylindrical body 9 and a slightly conical distal end 10, which has a similar cross section to the drive recess 3.

In a preferred embodiment, the cross-section of the distal end 10 is larger in the proximal portion 11 of the distal end 10 than in the distal portion 12. This type of slightly conical shape is especially advantageous, because it allows the distal end 10 to be easily pushed into the drive recess 3 and easily withdrawn from the screw when it has been fully inserted. The distal end 10 of the instrument 8, because of its similarly shaped cross-section, fits tightly in the drive recess 3 of the screw when fully inserted into the screw.

As shown in FIG. 4, there can be a circular nib 13 at the distal end 10 of the inserter 8, that is useful when inserting screws having a central bore. The nib 13 has a cross section that is similar to that of a central bore in the screw to be inserted and is designed to slide tightly into the central bore 7 below the drive recess 3. This tight fit stiffens the screw/instrument combination thereby making it more stable during insertion.

Screws of the present invention may also comprise at least one longitudinal groove on the outer surface of the head. These screws may be inserted using a screwdriver having a corresponding projection, which penetrates into the aforementioned groove. In this way the torque against the screw, when turning the screw, can be dispersed to the inside and outside of the screw, which may improve the torsional resistance of the screw.

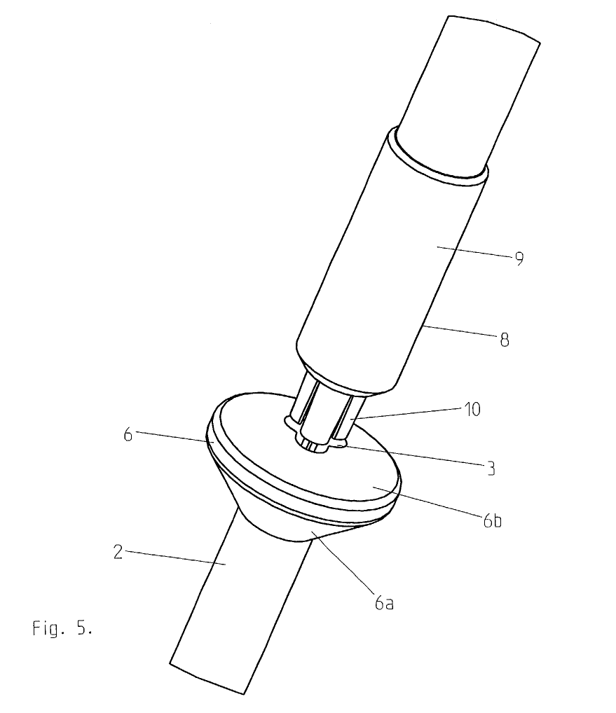

FIG. 5 shows embodiments of a surgical screw and an inserter instrument of the present invention in contact with each other. The distal end 10 of the instrument 8 is being pushed into the recess 3. When it is fully inserted into the recess 3, a firm grip is achieved between the screw and the inserter, thus enabling a reliable implantation of the screw.

The present invention and its applicability are described in more detail by means of the following nonlimiting examples.

### EXAMPLES

### Example 1

Bioabsorbable screws were machined from an oriented polymer composite. Two types of screws were made.

1. A screw which can be held by a driver placed outside of a flat head. The dimensions of the screw were: length 50 mm, diameter of the shank part=3.5 mm (=minimum diameter of the thread), maximum diameter=4.5 mm (thread throughout the whole shaft part of the screw), maximum diameter of the head=8 mm.

2. A screw in accordance with current invention which can be held by a driver by pushing the distal end of the driver into a recess having a cross-sectional shape of a cloverleaf. The dimensions of the screw were: length 50 mm, diameter of the shaft part=3.5 mm (=minimum diameter of the thread), maximum diameter=4.5 mm (thread throughout the whole shaft part of the screw), maximum diameter of the head=9.5 mm.

The torsional strengths of the two types of screws were measured by connecting the screw and driver together, by affixing the threaded portion of the screw to a device for measuring torsional strength, and by turning the driver and screw in opposite directions around the longitudinal axis of the screw until it started to break.

The torsional strength of the screw 2 in accordance with the invention was 20% better than torsional strength of screw 1 despite the fact that the screw 1 has 35% smaller head than the screw 2.

### Example 2

Bioabsorbable screws were machined from oriented polymer composite. Three types of recesses in the heads of the screws were made.

1. Square hole, wherein the length of the each four sides was 3 mm

2. Hex socket, wherein the length of the each six sides was 2 mm

3. Three lobed cloverleaf, wherein the radius of the leaves was 0.75 mm

The lengths of the circumferences of the different drive recesses were equal in each case. The drive recess having the shape of the cloverleaf had the smallest cross-sectional area, the square hole was 1.3 times larger and the hex socket was 1.5 times larger than the cloverleaf design. The best grip was achieved by the cloverleaf design although the cross-sectional area of the cloverleaf design was the smallest.

What is claimed is:

1. A surgical screw which is at least partially bioabsorbable in vivo comprising:
an elongated shank, at least a portion of said shank comprising threads; and
a head comprising a proximal surface that is substantially perpendicular to the longitudinal axis of the shank,
said shank protruding distally from said head,
said head comprising a recess in said proximal surface, said recess having a cross-section with a rotational symmetry around the longitudinal axis of the shank, said recess comprising an odd number of rounded lobes extending away from the center of the head.

2. A screw according to claim 1, wherein said lobes are selected from the group consisting of ovals, circles, and rectangles with rounded edges.

3. A screw according to claim 1, wherein said recess is formed by a process selected from the group consisting of machining, thermoforming, and machining combined with thermoforming.

4. A screw according to claim 1, wherein at least a portion of said screw comprises self-reinforced material.

5. A screw according to claim 1, wherein said head further comprises a distal surface that tapers towards said shank.

6. A screw according to claim 1, wherein said screw further comprises a bore extending from said recess towards said shank.

7. A screw according to claim 6 wherein said bore extends the entire length of said screw.

8. A screw according to claim 1 wherein said recess extends the entire length of said screw.

9. A surgical screw which is at least partially bioabsorbable in vivo comprising:
an elongated shank, at least a portion of said shank comprising threads; and
a head comprising a proximal surface that is substantially perpendicular to the longitudinal axis of the shank,
said shank protruding distally from said head,
said head comprising a recess in said proximal surface,
said recess having a cross-section with a rotational symmetry of 120 degrees around the longitudinal axis of the shank,
said recess having three rounded lobes extending away from the center of the head, said lobes forming the cross-sectional shape of a cloverleaf.

10. A screw according to claim 9 wherein said screw further comprises a bore extending from said recess towards said shank.

11. A screw according to claim 10, wherein said bore extends the entire length of said screw.

12. A surgical screw which is at least partially bioabsorbable in vivo comprising:
an elongated shank, at least a portion of said shank comprising threads; and
a head comprising a proximal surface that is substantially perpendicular to the longitudinal axis of the shank,
said shank protruding distally from said head,
said head comprising a plurality of recesses in said proximal surface,
said recesses being located in said head so as to have a rotational symmetry around the longitudinal axis of the shank,
said recesses having curved shapes.

13. A screw according to claim 12, wherein said recesses have shapes selected from the group consisting of ovals, circles, and rectangles with rounded edges.

14. A surgical screw system comprising:
a surgical screw which is at least partially bioabsorbable in vivo and an inserter for said screw,
wherein said screw comprises:
an elongated shank, at least a portion of said shank comprising threads; and
a head comprising a proximal surface that is substantially perpendicular to the longitudinal axis of the shank,
said shank protruding distally from said head,
said head comprising a recess in said proximal surface,
said recess having a cross-section with a rotational symmetry around the longitudinal axis of the shank,
said recess having three rounded lobes extending away from the center of the head,
said lobes forming the cross-sectional shape of a cloverleaf;
and wherein said inserter for said screw comprises:
an elongated body having a distal end,
said distal end comprising three rounded lobes forming the shape of a cloverleaf in cross section,
said lobes having a rotational symmetry around the longitudinal axis of said body, at least a portion of said distal end having a cross section substantially the same as the cross section of said recess in said screw head;
wherein said distal end is capable of slidable engagement with said recess in said screw head.

15. The surgical screw system of claim 14,
wherein the cross section of said distal end gets progressively smaller towards the distal tip of said distal end.

16. The surgical screw system of claim 15,
wherein said screw further comprises a bore exending from said recess towards said shank; and said inserter further comprises a distal protrusion protruding from said distal end, said distal protrusion having a cross section substantially the same as the cross section of said bore;
wherein said distal protrusion is capable of slidable engagement with said bore.
